(19) United States
(12) Patent Application Publication
(10) Pub. No.: US 2025/0332336 A1
(43) Pub. Date: Oct. 30, 2025

(54) INTEGRATED NEGATIVE PRESSURE WOUND THERAPY DEVICE WITH DWELL, OBSERVABILITY, AND INSTILLATION

(71) Applicants: Songlin Yang, Shanghai (CN); Chenxi Yang, Acton, MA (US)

(72) Inventors: Songlin Yang, Shanghai (CN); Chenxi Yang, Acton, MA (US)

(21) Appl. No.: 18/978,313

(22) Filed: Dec. 12, 2024

Related U.S. Application Data
(60) Provisional application No. 63/639,128, filed on Apr. 26, 2024.

(51) Int. Cl. A61M 1/00

(52) U.S. Cl. CPC A61M 1/915; A61M 1/912; A61M 1/92

(57) ABSTRACT

A negative pressure wound therapy device comprises a cover, and a base. The cover includes an inner side and an outer side. One or more connectors are disposed on the outer side. The base includes an upper side and an under side. The device further includes a plurality of posts in between the cover and the base. The base is configured with a plurality of orifices. The cover and the base are integrated to form a pocket. At least one of the one or more connectors is a drainage connector for draining liquid out of the pocket. The wound therapy device is configured to connect to a vacuum machine to drain the pocket through the drainage connector. The wound therapy device is efficient and durable, safe and comfortable, and is convenient to use and manage.

### RELATED APPLICATIONS

This application claims priority U.S. Provisional Application 63/639,128 filed on Apr. 26, 2024, the entire content of which is incorporated herein in its entirety.

### FIELD OF THE TECHNOLOGY

The present disclosure relates generally to medical devices, and more specifically to negative pressure wound therapy devices.

### BACKGROUND

In the field of wound care technologies, negative pressure wound therapy (NPWT) has become a popular and effective treatment modality. NPWT is a globally recognized wound-treating technique. NPWT relies on a vacuum machine to facilitate liquid drainage and debridement from the wound. NPWT can be used in a number of settings, for example, grafting, preparing surgical wound sites for closure, or treating surgical wounds, traumatic wounds, and complex ulcerative wounds such as diabetic, pressure and venous stasis induced wound, and wounds involving exposed bone and orthopedic implants. Meanwhile, NPWT, when used properly, promotes nursing and healing, and stimulates tissue growth.

Existing NPWT devices use open-pore foam sponge. The foam sponge used in NPWT devices is often made of black polyurethane soaked in ether or white polyvinyl. There are several drawbacks with existing NPWT devices. Foam sponge is compressible and the pores inside a foam sponge collapse and shrink in size irreversibly when compressed, blocking the pathway of liquid exudates and solid wastes. The blocked liquid exudates and solid wastes may accumulate inside the foam sponge and form a blocked layer at or near the bottom of the foam sponge that leads to device malfunction.

Further, the small pore size of a sponge, along with the circuitous conduction pathways formed by the pores, leads to low permeability and negative pressure loss. Also, foam sponge is opaque. Existing NPWT devices do not allow doctors to observe the wound underneath the device without removing the device first.

Other drawbacks of existing NPWT devices include that foam sponge is made of plastic that can cause irritation to body tissue inside the wound. Also, foam sponge is not durable due to high brittleness and can break off during removal, especially when new body tissue has grown into the pores of the foam sponge. The broken bits from the foam sponge may be left in the healed wound.

Lastly, existing NPWT devices require in situ assembling, therefore not convenient to use and prone to failure.

### SUMMARY

Accordingly, it is an objective of the present application to disclose an improved negative pressure wound therapy (NPWT) device. Compared to existing NPWT devices, the NPWT devices disclosed herein are integrated devices with reticulated structure simulating the porous structure of a sponge, made from durable, biocompatible, or medical grade material or materials (e.g., silicone, silicone rubber, polymer, etc.) instead of foam sponge. Unlike existing NPWT devices, the NPWT devices disclosed herein do not collapse under negative pressure and do not become blocked, provide extended patency and dwell. This is because the material or materials used in the devices disclosed herein offer greater strength, stability, and durability than the plastic nature of sponge foam, and the intentionally designed reticulated structure offers wider and simpler pathways than the natural structure of sponge foam. The NPWT devices disclosed herein can be made of transparent or translucent materials, allowing doctors to observe the condition of the wound underneath. Therefore, the improved NPWT devices disclosed herein require less frequent removal and replacement, hence longer patency and dwell. Unlike foam sponge, the material or materials used in the NPWT devices disclosed herein are safe and comfortable to the touch. The materials will not cause irritation to open wounds or injured skin, which is not ideal. The materials are pliable and sturdy, not easy to break off during removal even when new tissue has become attached. The NPWT devices in the present disclosure do not require in situ assembly, are easy to use and clean, and are not prone to breakage or failure.

In some embodiments, a wound therapy device comprises a cover and a base. The cover has an inner side and an outer side. One or more connectors are disposed on the outer side. The base has an upper side and an under side. The base is configured with a plurality of orifices. The cover and the base are integrated to form a pocket in between the inner side of the cover and the upper side of the base. The wound therapy device further comprises a plurality of posts that are inside the pocket. In one embodiment, the posts form a reticulated structure. At least one of the one or more connectors is a drainage connector for drawing liquid exudates and solid wastes out of the pocket. In one embodiment, the base and/or the cover are membranous.

In some embodiments, optionally, at least one of the one or more connectors is an instillation connector for injecting a liquid into the pocket. The liquid may include, but not limited to, a rinsing liquid, a medicated liquid, or the like.

In some embodiments, the size and shape of the base and/or the size and shape of the cover are customizable to fit the wound.

In some embodiments, the plurality of posts form a reticulated structure inside the pocket to prevent the pocket from collapsing and to create a pathway. The plurality of posts are configured to prevent the pocket from collapsing and to create a pathway for the viscous liquid and/or tissue fragments to move inside the pocket and exit the pocket via one or more drainage connectors. For example, in some embodiments, the plurality of posts are attached to the upper side of the base and/or attached to the underside of the cover to form an integrated device. The plurality of posts are configured to create space between the cover and the base and among the posts. The reticulated structure formed by the posts creates pathways to allow gas, rinsing liquid, viscous body liquid and/or tissue fragments to pass through.

In some embodiments, the size of the orifices on the base is configured to allow viscous liquid and/or tissue fragments to pass through the base and enter the pocket. The size of the orifices may range from 1 mm to 10 mm. The plurality of orifices may be evenly or unevenly distributed.

In some embodiments, the height of the plurality of posts is uniform. In some embodiments, the height of a post may vary depending on the position of the post. In some embodiments, the height of a post may range from 1 mm to 10 mm approximately or substantially, a diameter of a post may range from 1 mm to 5 mm approximately or substantially, and a distance between two posts in the plurality of posts may range from 1 mm to 10 mm approximately or substantially. In some embodiments, the shape of the posts can be cylindrical, square, polygonal, spherical, etc.

In some embodiments, the drainage connector is configured to connect to a vacuum machine to drain the pocket by creating a negative pressure in the pocket. The drainage connector is configured to collect returned rinsing liquid, viscous body liquid and/or tissue fragments out of the pocket. The size of a drainage connector and the size of an instillation connector can be the same or different.

In some embodiments, the cover and the base are made of a same material. For example, the cover and the base are made of silicone or silicone rubber. The base and/or the cover may be membranous. When the device is membranous and made of silicone or silicone rubber, the device offers better histocompatibility and comfort.

In some embodiments, a negative pressure wound therapy device may include a base, a cover, and a middle layer. The NPWT device further includes a plurality of posts disposed in between the base and the middle layer and a plurality of posts disposed in between the middle layer and the cover. The middle layer includes one or more orifices to allow viscous liquid and solid wastes to pass through the layer. The middle layer may be made of a same or different material than the base or the cover. In one embodiment, the NPWT device may include more than one middle layer.

In some embodiments, a wound therapy device includes a cover, a base, a plurality of posts in between the cover and the base, and a pocket formed between the cover and the base. The cover is configured with one or more drainage connectors and one or more instillation connectors. The base is configured with a plurality of orifices. The pocket is supported by a plurality of posts to prevent the pocket from collapsing. The plurality of orifices on the base are configured to allow viscous body liquid and/or tissue fragments to pass through the base into the pocket.

In one embodiment, the posts are made of silicone or silicone rubber. The posts can be made of other similar materials, such as polyurethane. The shape of the posts can be cylindrical, square, polygonal, spherical, etc. In one embodiment, the entire wound therapy device is made of silicone or silicone rubber. In one embodiment, the cover and the base are membranous.

In some embodiments, the posts may be attached to the base and the cover, and the wound therapy device becomes an integrated device. In other embodiments, the posts may be attached to either the base or the cover.

In one embodiment, the drainage connector is configured to connect to a vacuum machine to create a negative pressure inside the pocket to empty the content of the pocket. In one embodiment, the cover is configured with at least one instillation connector to allow injection of a rinsing liquid or medicated liquid.

In some embodiments, the size and shape of the base in the wound therapy device are customizable. In some embodiments, the size and shape of the cover in the wound therapy device are customizable. In some embodiments, both the size and shape of the cover and the base in the wound therapy device are customizable.

### BRIEF DESCRIPTION OF THE DRAWINGS

These and other features of the present disclosure will become readily apparent upon further review of the following specification and drawings. In the drawings, like reference numerals designate corresponding parts throughout the views. Moreover, components in the drawings are not necessarily drawn to scale, the emphasis instead being placed upon clearly illustrating the principles of the present disclosure.

FIG. 1 is an illustration of an example configuration used in negative pressure wound therapy.

FIG. 2A-FIG. 2D illustrate different embodiments of the wound therapy device disclosed in the present application.

FIG. 3 illustrates a top view of a wound therapy device of the present application.

FIG. 4 illustrates another top view of another embodiment of a wound therapy device of the present application.

FIG. 5 illustrates the side view of two embodiments of a wound therapy device of the present application.

FIG. 6 illustrates a bottom view of a wound therapy device of the present application.

FIG. 7 illustrates another embodiment of a wound therapy device of the present application.

FIG. 8 illustrates an example application of a wound therapy device on a wounded limp.

### DETAILED DESCRIPTION

Embodiments of the disclosure are described more fully hereinafter with reference to the accompanying drawings, in which preferred embodiments of the disclosure are shown. The various embodiments of the disclosure may, however, be embodied in many different forms and should not be construed as limited to the embodiments set forth herein. Rather, these embodiments are provided so that this disclosure will be thorough and complete, and will fully convey the scope of the disclosure to those skilled in the art.

Embodiments set forth herein describe a wound therapy device that is more efficient at discharging tissue fragments and draining exudates, more comfortable to the touch, and easier to operate than exiting negative pressure wound therapy (NPWT) devices.

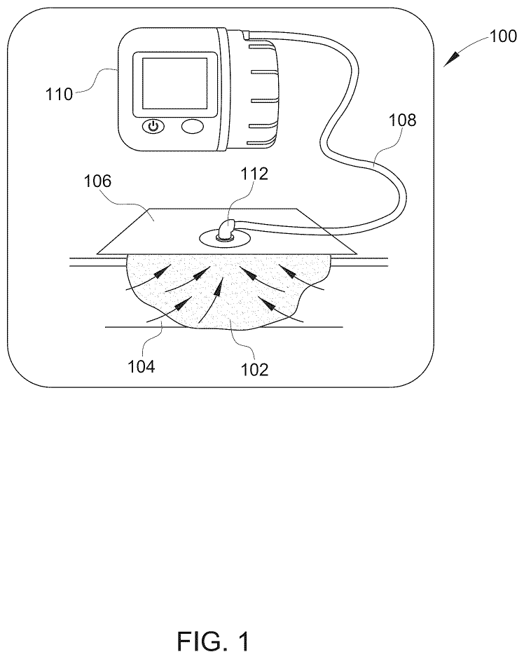

In referring to FIG. 1, an NPWT device 100 is shown to include a foam sponge 102, a dressing 104, a film seal 106, a tubing 108, and a vacuum machine 110. The foam sponge 102 may be made of black polyurethane or white polyvinyl and may be sterilized by soaking into alcohol or ether. The foam sponge 102 needs cutting to match the unique shape of each wound. The foam sponge 102 is used as filler inside the dressing 104. The dressing 104 may be a semi-occlusive adhesive drape, and, with the customized foam sponge 102 filled in, the dressing 104 can be applied to the wound. A film seal 106 is applied on the dressing to seal the wound. The dressing 104 is connected to a vacuum machine 110 via a tube 108. One end of the tube 108 is connected to the vacuum machine 110 and the other end of the tube 108 is inserted into a connector 112 on the film seal 106. When the vacuum machine 110 is powered on, it draws out the air inside the foam sponge 102 and creates a negative pressure environment inside the foam sponge 102. The foam sponge 102 absorbs viscous liquid exudates and solid wastes such as tissue fragments, discharges, and dead cells. The vacuum machine 110 draws the viscous liquid exudates and solid wastes from inside the foam sponge 102 into the vacuum machine 110, to keep the wound clean. The arrows illustrate pathways taken by the liquid exudates and solid wastes when being drawn out of the foam sponge 102 into the tube 108 through the connector 112.

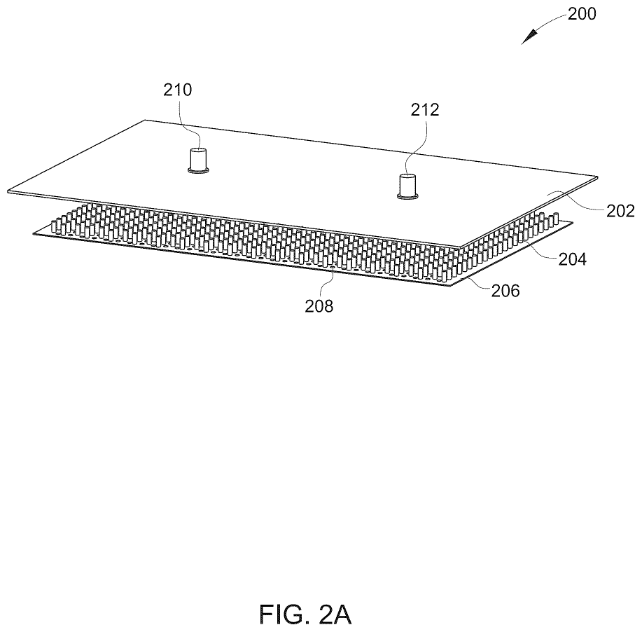

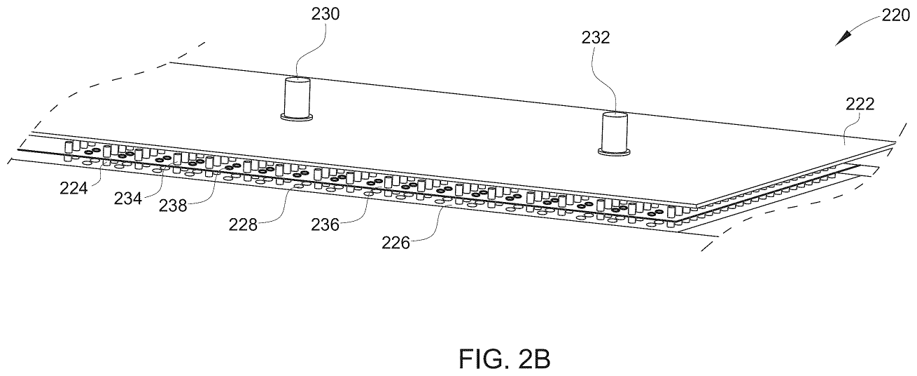

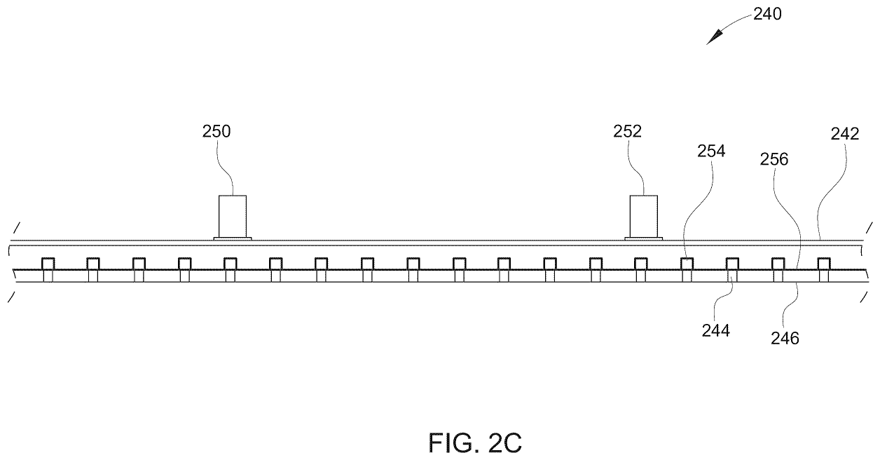

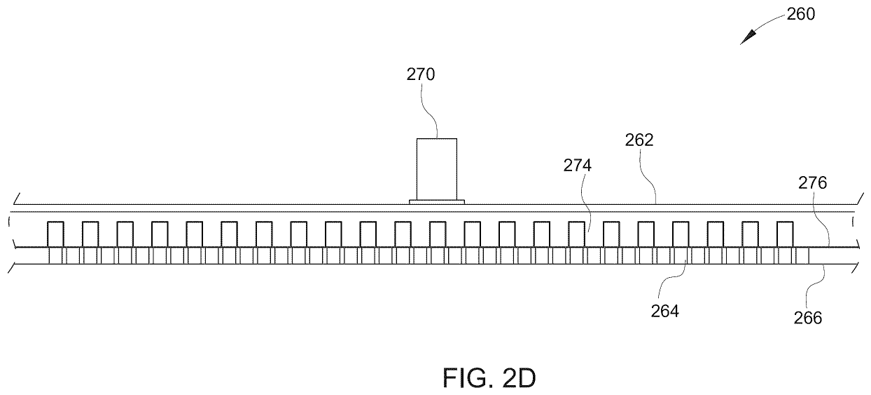

Under the negative pressure, the foam sponge 102 becomes compressed and the pores in the foam sponge 102 become smaller or even closed, blocking the pathways. As the pathways become impassable, the liquid exudates and solid wastes start to accumulate inside the foam sponge 102 and may form a blocked layer at or near the bottom of the foam sponge 102. The NPWT device 100 becomes less effective and needs to be replaced. However, frequent removing and replacement of the NPWT device 100 causes discomfort and pain in patients, requires attention from medical professionals, and wastes medical resources. The present application discloses an improved wound therapy device 200 (FIG. 2A) that overcomes the above-described drawbacks of the NPWT device 100. The device 200 is more effective as therapeutic treatment, easier to operate and manage by health care providers more comfortable for the patients, more stable in structure, without irreversible collapse and blockage. The device 200 does not leave residuals in wound tissues. The device 200 can create a better healing environment and achieve higher health and economy efficacy.

In referring to FIG. 2A, a wound therapy device 200 comprises a cover 202 and a base 206. The cover 202 has an inner side and an outer side. On the outer side of the cover 202, one or more connectors are disposed. At least one of the one or more connectors is a drainage connector 210. Optionally, one of the one or more connectors is an instillation connector 212. The drainage connector 210 is configured to connect via a tube to a vacuum machine (not shown in FIG. 2A). When the vacuum machine is turned on, solid wastes and viscous liquid are drawn out of the device 200 through the drainage connector 210. When available, the instillation connector 212 can be configured to connect to a supply apparatus. For example, a rinsing liquid or medicated liquid can be injected into the device 200 through the instillation connector 212.

The base 206 has an upper side and an under side. On the upper side of the base 206, a plurality of posts 204 are disposed. The base 206 is configured with one or more orifices 208. The cover 202 and the base 206 can be integrated to form a pocket in between the inner side of the cover 202 and the upper side of the base 206.

In the present application, how the cover 202 and the base 206 are integrated to form a pocket is not limited. For example, the edges of the cover 202 and the base 206 can be equipped with adhesive strips with removable cover. After the cover is removed and the adhesive strips are exposed, the cover 202 and the base 206 can be pressed together to form a sealed pocket. For another example, when the cover 202 and the base 206 are part of a seamlessly integrated assembly made of a same material, no assembling or cutting is required before the device 200 is applied to a wound. For yet another example, the cover 202 and the base 206 can be made of different materials.

In some embodiments, the cover 202 and the base 206 are both made of a same material, such as silicone or silicone rubber. When made of silicone or silicone rubber, the cover 202 has greater strength than the commonly used semi-occlusive adhesive drape so the cover 202 is less prone to damage or leakage. No frequent replacement is needed as compared to existing NPWT devices. When the base 206 is membranous and is made of silicone or silicone rubber, the base 206 provides more comfort and breathability when the device is laid on top of an open wound. Therefore, the device 200 offers better histocompatibility and comfort than existing NPWT devices.

However, not limited to silicone or silicone rubber, the device 200 can be made of any suitable material or materials. Materials having properties similar to silicone, for example, polymer, can be used to manufacture the device 200. In some embodiments, the cover 202 and the base 206 may be made of different materials. When in use, the underside of the base 206 is in touch with the skin or wound tissue while the outer side of the cover 202 is not. The requirements on the material of the cover 202 and of the base 206 can therefore different. For example, in one embodiment, the base 206 may be made of silicone or other synthetic materials that are medical grade or biocompatible, while the cover 202 may be made of silicone, polyethylene, or polyurethane. For another example, the base 206 is membranous and made of silicone, while the cover 202 is made of a different material with a different structure.

In the present application, the cover 202 and the base 206 can have various shapes such as circle, ellipse, square, rectangle, etc. The device 200 can also be customized to fit the shape of the wound. For example, the size of the device 200 and/or the shape of the device 200 can be customized to fit the wound. In some embodiments, the cover 202 is larger than the base 206 to seal the pocket directly. The shape of the base 206 can be the same as or different than the shape of the cover 202.

The base 206 is configured with one or more orifices 208. In some embodiments, the size of the orifices ranges from 1 mm to 10 mm. The orifices 208 provide the viscous liquid and solid wastes an entrance into the pocket. A plurality of posts 204 are disposed inside the pocket. In some embodiments, the plurality of posts 204 are attached to the upper side of the base 206. The posts 204 may be made of the same material as the base, for example, silicone. The posts 204 may be made of a different material than that of the base. In some embodiments, the plurality of posts 204 are attached to the inner side of the cover 202. And in some embodiments, the plurality of posts 204 are attached to both the upper side of the base 206 and the inner side of the cover 202.

The posts 204 are of a certain height and the height may be uniform or varied. The shape of the posts can be cylindrical, square, polygonal, spherical, etc. The posts 204 function like the sponge fiber in the foam sponge 102 and create space similar to sponge pores. The posts 204 prevent the pocket formed in between the cover 202 and the base 206 from collapsing and ensure that sufficient space exists between the cover 202 and the base 206 to allow free flow of air and discharges when the device 200 is connected to a vacuum machine. In FIG. 2, the posts 204 are shown to be attached to the upper side of the base 206. In other embodiments, the posts 204 may be attached to the inner side of the cover 202 or attached to both the cover 202 and the base 206. The plurality of the posts 204 create pathways for the viscous liquid and solid wastes to pass through the pocket. The plurality of the posts 204 may form a reticulated structure or other structures inside the pocket.

FIG. 2B-FIG. 2C illustrate embodiments that include a middle layer between the cover 202 and the base 206. In FIG. 2B, an NPWT device 220 includes a cover 222, a base 226, and a middle layer 236. On top of the cover 222, there are a drainage connector 230 and an instillation connector 232. A plurality of posts 224 are disposed in between the base 226 and the middle layer 236. A plurality of posts 234 are disposed in between the middle layer 236 and the cover 222. On the base 226, a plurality of orifices 228 are distributed. On the middle layer 236, a plurality of orifices 238 are distributed. In FIG. 2B, the middle layer 236 and the base 226 are configured identically with orifices and posts. However, in other embodiments, the orifices on the middle layer 236 and the orifices on the base 226 may be configured differently, in terms of density, size, and shape.

FIG. 2C and FIG. 2D show two NPWT devices 240 and 260 that also include a middle layer, 256 and 276, respectively. In FIG. 2C, the NPWT device 240 is configured with two connectors, 250 and 252. A plurality of posts 254 are disposed in between the middle layer 256 and the cover 242 and a plurality of posts 244 are disposed in between the base 246 and the middle layer 256. The plurality of posts 254 are of different shape and size than the plurality of posts 244.

In FIG. 2D, the NPWT device 260 is configured with one connector 270. A plurality of posts 264 are disposed in between the middle layer 276 and the cover 262. A plurality of posts 274 are disposed in between the cover 266 and the middle layer 276. The plurality of posts 274 are of different shape, density, and size than the plurality of posts 264. In comparing FIG. 2D with FIG. 2C, the densities of posts 274 and 264 in FIG. 2D is higher than the densities of posts 254 and 244 in FIG. 2C. The size and shape of the posts 274 are different than the size and shape of the posts 264. Further the posts 264 are attached to both the middle layer 276 and the base 266, while the posts 274 are attached to the middle layer 276 only.

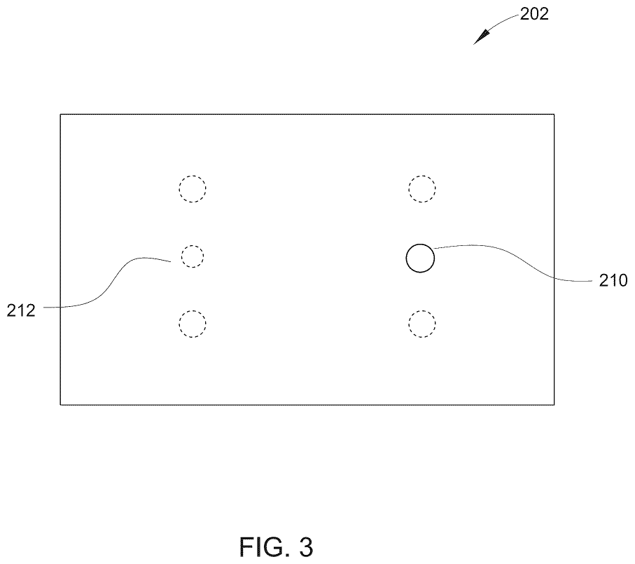

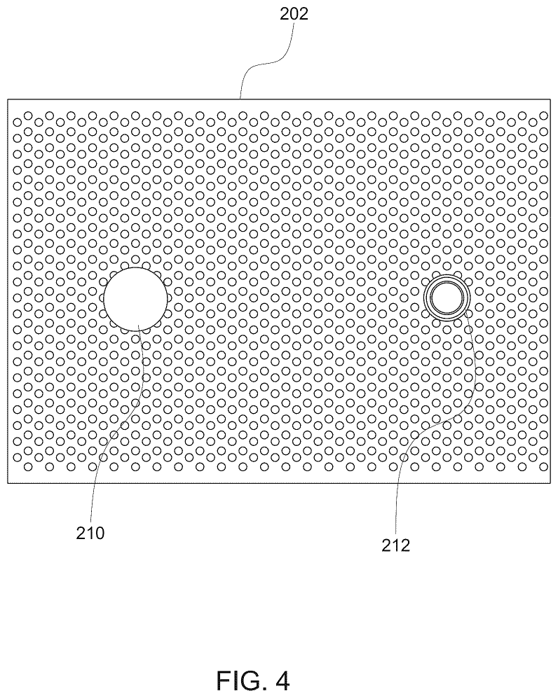

The device 200 is configured to connect to a vacuum machine via a drainage connector 210 located on the outer side of the cover 202. FIG. 3 illustrates a top view of the device 200, i.e., the outer side of the cover 202. As shown in FIG. 3, one or more connectors are disposed on the cover 202. The connector 210 is a drainage connector and the connector 212 is an instillation connector. The instillation connector 212 is optional. In some embodiments, the instillation connector 212 is absent. FIG. 4 illustrates an enlarged view of the drainage connector 210 and the instillation connector 212. Both connectors are disposed on the outer side of the cover 202. When the device 200 is in use, the drainage connector 210 is normally connected to a vacuum machine. When the vacuum machine is turned on, the liquid exudates and solid wastes accumulated in the pocket of the device 200 are drained through the drainage connector 210. The instillation connector 212 is equipped with a cap. The cap is closed when the instillation connector 212 is not in use. The cap can be opened to allow the instillation connector 212 to connect to a medicine supply apparatus (not shown in FIG. 4). The medicine supply apparatus can inject rinsing liquid or medicated liquid into the pocket through the instillation connector 212.

In FIG. 4, the drainage connector 210 is relatively large in size as compared to the instillation connector 212. This is because the substances that pass through the drainage connector 210 may contain solid particles and debris of large diameters. In comparison, only pure liquid passes through the instillation connector 212. In some embodiments, the drainage connector and the instillation connector may be of the same size.

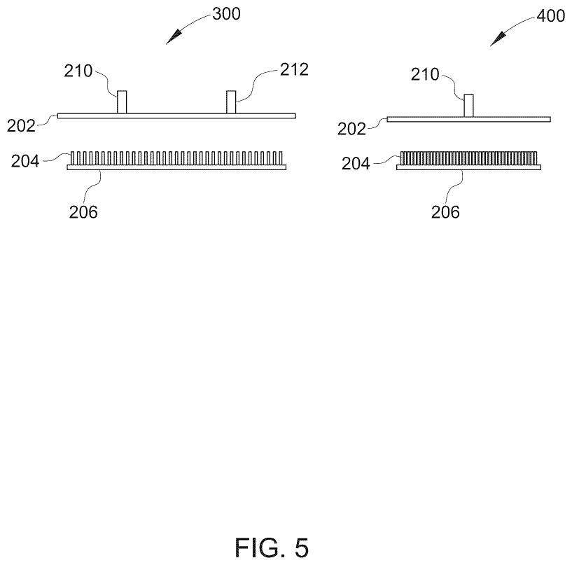

FIG. 4 is a top view of the device 200. The reticulated structure shown in FIG. 4 is a view from the top of the posts through a translucent, transparent, or semi-transparent/translucent cover 202. FIG. 5 illustrate two side views of the posts in two embodiments of a wound therapy device, device 300 and device 400.

In the device 300, the posts 204 disposed on the upper side of the base 206 are relatively tall and are spaced farther apart. The cover 202 are configured with a drainage connector 210 and an instillation connector 212. In comparison, the device 400 is of a smaller size. The posts 204 disposed on the upper side of the base 206 are relatively small and are more densely arranged. The cover 202 in the device 400 is configured with only a drainage connector 210. In practice, the device 300 may be selected for use in treating larger and more traumatic injuries, while the device 400 is more suitable for smaller wounds or less severe injuries.

In some embodiments, the posts 204 are of uniform size and/or uniform density. The post height ranges from approximately 1 mm to 10 mm. The diameter of a post ranges from approximately 1 mm to 5 mm. The distance between two neighboring posts ranges from approximately 1 mm to 10 mm.

In some embodiments, the posts 204 are not uniform in size. For example, the post height may vary depending on whether the posts are close to the edge or near the center. For another example, the width or diameter of a post may vary depending on the position of the posts. The distance between two neighboring posts is not required to be the same as well. The shape of the posts 204 may be cylindrical, spherical, polygonal, square, etc.

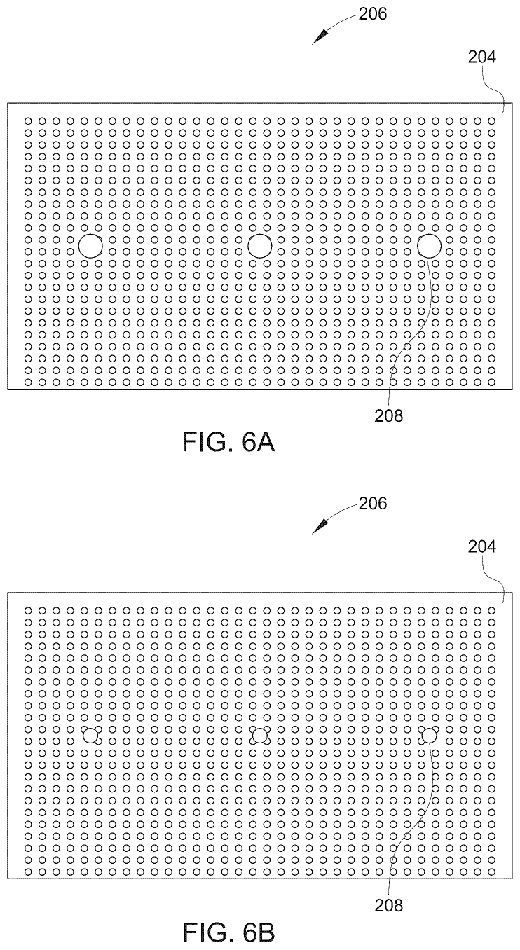

FIG. 6A illustrates a bottom view of the device 200, i.e., the underside of the base 206. The base 206 are configured with three orifices 208. It is noted that the number of orifices on the base 206 can be as many as needed. The orifices 208 are evenly distributed on the base 206. In some embodiments, the orifices 208 may be unevenly distributed on the base 206, for example, more concentrated in the middle than on the periphery. When the underside of the base 206 is placed on a wound, tissue fragments, exudates, blood, etc. from the wound can enter the pocket of the device 200 through the orifices 208. In some embodiments, the size of the orifices ranges from 1 mm to 10 mm. But the size of the orifices is not limited herein and can vary in accordance with different medical needs. FIG. 6B illustrates a bottom view of another embodiment of the device 200. In FIG. 6B, the base 206 is configured with a plurality of orifices 208 that are smaller in size but more numerous than the orifices 208 in FIG. 6A.

In some embodiments, the device 200 may be configured with an instillation connector 212 (see FIG. 4) that is connected to a medicine supply apparatus. Rinsing liquid or medicated liquid is injected into the pocket of the device 200 via the instillation connector 200. The rinsing liquid or medicated liquid can reach the wound through the orifices 208.

In FIG. 6. the reticulated structure is a view of the posts 204 through the translucent, transparent or semi-transparent/translucent base 206.

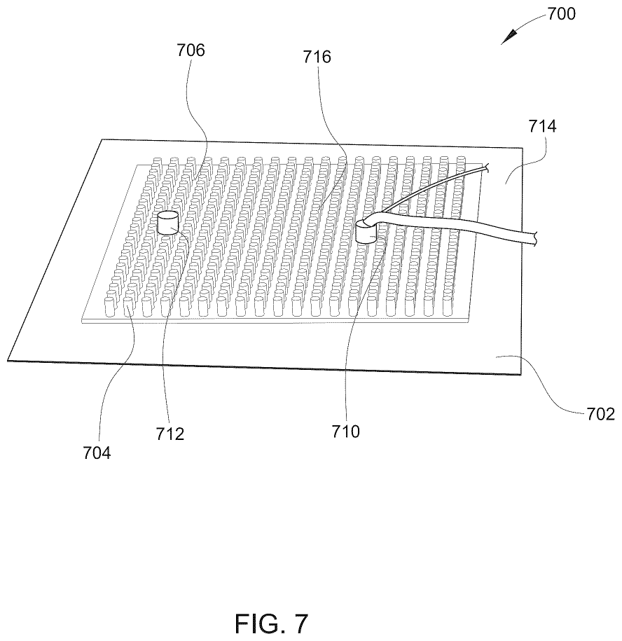

FIG. 7 illustrates another embodiment of a wound therapy device 700. The device 700 comprises a cover 702, a base 706, and a pocket formed between the cover 702 and the base 706. A plurality of posts 704 are disposed in between the cover 702 and the base 706. The cover 702 is configured with a drainage connector 710 and an instillation connector 712. In some embodiments, the cover 702 may be configured with more than one drainage connector 710 or more than one instillation connector 712. In one embodiment, one or more additional rinsing tubes 714 may be added to the drainage connector 710 to clear occasional clots in the connector 710. The base 706 is configured with a plurality of orifices that are not shown. The pocket 716 formed between the cover 702 and the base 706 is supported by a plurality of posts 704. The posts 704 prevents the pocket 716 from collapsing. The posts 704 are spaced apart from each other, leaving room for liquid exudates and solid wastes to pass in between the posts 704. The posts 704 may be uniform or non-uniform in size or in shape. The height of the posts 704 determines the size of the pocket 716.

In referring to FIG. 7, it is noted that the cover 702 is larger than the base 706. The cover 702 forms a tight cover of the wound and the surrounding area of the wound, under the negative pressure created by the vacuum machine. In some embodiments, a film is added over the cover 702 to create a better seal of the pocket 716. When the cover 702 and the base 706 are made of translucent, transparent or semi translucent/transparent materials, the device 700 provides excellent observability, which allows medical personnel to observe the condition of the wound without removing the device 700, improving patency and dwell of the device 700.

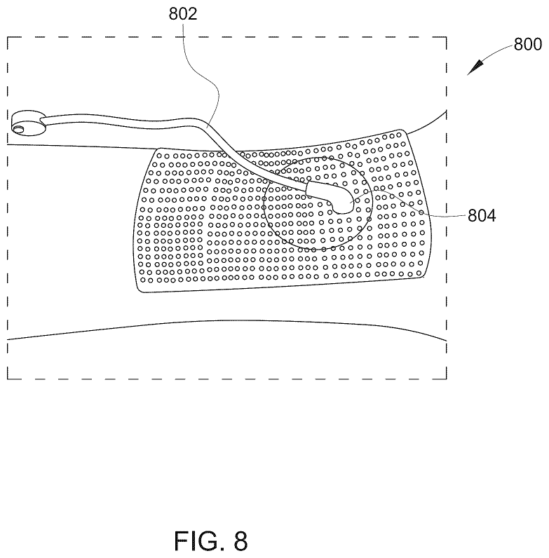

FIG. 8 illustrates a wound therapy device 800 applied onto a wounded limp. The device 800 is connected to a tube 802 via a drainage connector 804. The tube 802 is connected to a vacuum machine that is not shown in the figure. Gas, viscous liquid, and tissue fragments from the wound can enter the orifices on the base, pass through the space around the posts, and eventually move out of the pocket from the drainage connector 804 on the panel into the tube 802. Silicone-made posts are strong enough to ensure the stability of the structure and the patency of the space, therefore can avoid obstruction and device failure.

Although the disclosure is illustrated and described herein with reference to specific embodiments, the disclosure is not intended to be limited to the details shown. Rather, various modifications may be made in the details within the scope and range of equivalents of the claims and without departing from the disclosure.

What is claimed is:

1. A device for wound therapy, comprising:
a cover having an inner side and an outer side, wherein one or more connectors are disposed on the outer side;
a base having an upper side and an under side, wherein the base is configured with a plurality of orifices; and
a plurality of posts;
wherein the cover and the base are integrated to form a pocket in between the inner side of the cover and the upper side of the base; and
wherein one of the one or more connectors is a drainage connector for draining the pocket.

2. The device of claim 1, wherein a size and a shape of the base are customizable and/or a size and a shape of the cover are customizable to fit a wound.

3. The device of claim 1, wherein the plurality of posts form a reticulated structure inside the pocket to prevent the pocket from collapsing and to create a pathway.

4. The device of claim 1, wherein at least one of the one or more connectors is an instillation connector for injecting a liquid into the pocket.

5. The device of claim 4, wherein the liquid injected into the pocket is one of the following:
rinsing liquid or medicated liquid.

6. The device of claim 1, wherein a size of the orifices on the base is configured to allow viscous liquid and/or tissue fragments to pass through the base and enter the pocket.

7. The device of claim 1, wherein a shape of the plurality of the posts can be cylindrical, square, polygonal, or spherical.

8. The device of claim 3, wherein a height of a post in the plurality of posts is configured to create a space between the cover and the base and among the posts, and wherein the space forms the pathway to allow gas, rinsing liquid, viscous body liquid and/or tissue fragments to pass through.

9. The device of claim 7, wherein a height of a post in the plurality of posts ranges from approximately 1 mm to 10 mm, a diameter of a post in the plurality of posts ranges from 1 mm to 5 mm substantially, and a distance between two neighboring posts ranges from approximately 1 mm to 10 mm.

10. The device of claim 1, wherein the drainage connector is configured to connect to a vacuum machine to drain the pocket by creating a negative pressure in the pocket.

11. The device of claim 10, wherein the drainage connector is configured to collect rinsing liquid, viscous body liquid and/or tissue fragments for draining out of the pocket.

12. The device of claim 1, wherein the cover or the base is made of silicone or silicone rubber.

13. The device of claim 12, wherein the base is membranous and made of silicon rubber to offer better histocompatibility and comfort.

14. The device of claim 1, wherein the plurality of posts are attached to the cover and the base to form an integrated device.

15. The device of claim 1, further comprising a middle layer in between the base and the cover, wherein the middle layer is configured with one or more orifices, and wherein a plurality of posts are disposed in between the middle layer and the cover to form a reticulated structure, and a plurality of posts are disposed in between the middle layer and the base to form a reticulated structure.

16. A wound therapy device, comprising:
a cover;
a base;
a plurality of posts in between the cover and the base; and
a pocket formed between the cover and the base;
wherein the cover is configured with one or more drainage connectors, and the base is configured with a plurality of orifices; and
wherein the pocket is supported by the plurality of posts to prevent the pocket from collapsing.

17. The wound therapy device of claim 16, wherein the plurality of orifices on the base are configured to pass liquid exudates and/or tissue fragments into the pocket.

18. The wound therapy device of claim 17, wherein the one or more drainage connectors are configured to connect to a vacuum machine to create a negative pressure inside the pocket to allow the pocket to drain off.

19. The wound therapy device of claim 18, wherein the cover is further configured with one or more instillation connectors and the one or more instillation connectors are configured to take injection of a rinsing liquid or medicated liquid.

20. The wound therapy device of claim 16, wherein a size and a shape of the base are customizable and/or a size and a shape of the cover are customizable according to a wound.
